(19) United States
(12) Patent Application Publication
(10) Pub. No.: US 2010/0151010 A1
(43) Pub. Date: Jun. 17, 2010

(54) MEDICAMENT IN A MULTILAYER FORM

(75) Inventors: Hans-Ulrich Petereit, Darmstadt (DE); Christian Meier, Darmstadt (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: ROEHM GMBH, Darmstadt (DE)

(21) Appl. No.: 11/569,731

(22) PCT Filed: May 18, 2005
(86) PCT No.: PCT/EP2005/005374

§ 371 Date: Nov. 28, 2006

(30) Foreign Application Priority Data
Jul. 23, 2004 (DE) 10 2004 035936.9

(51) Int. Cl. A61K 9/14; A61K 31/606; A61K 31/196; A61K 31/573; A61K 31/58; A61K 38/43; A61K 38/00; A61K 39/00; A61K 39/395; A61K 38/54; A61K 38/28; A61K 38/22; A61K 31/7088; A61K 38/23; A61K 38/21; A61K 38/20; A61K 38/29; A61K 38/26; A61K 31/352; A61K 38/08; A61K 38/11; A61K 9/20; A61K 9/48; B05D 3/02

(52) U.S. Cl. 424/452; 424/497; 514/166; 514/565; 514/179; 514/174; 424/94.1; 514/2; 424/184.1; 424/130.1; 424/94.21; 514/3; 514/12; 514/44 R; 424/85.1; 424/85.4; 424/85.2; 514/456; 514/15; 424/465; 427/2.14

(57) ABSTRACT

The invention relates to a medicament in a multilayer form, containing a) a core with a pharmaceutical agent, b) an inner coating, 50 to 95 percent by weight of which arc composed of a (co)polymer comprising 95 to 100 percent by weight of radically polymerized vinylic monomers with neutral side groups and 0 to 5 percent by weight of monomers with anionic side groups, c) an outer coaling made of a copolymer comprising 75 to 95 percent by weight of radically polymerized C₁ to C₄ alkyl esters of acrylic acid or methacrylic acid and 5 to 25 percent by weight of (meth)acrylate monomers with an anionic group in the alkyl radical. Said medicament further contains 5 to 30 percent by weight of common pharmaceutical auxiliaries, particularly emollients. The inventive medicament is characterized in that the inner coaling contains 5 to 50 percent by weight of common pharmaceutical auxiliaries which are no expanding agents while the amount of expanding agents provided is less than 5 percent by weight.

The invention relates to a multilayer pharmaceutical form composed of a core with an active pharmaceutical ingredient, an inner polymer coating and an outer polymer coating.

### PRIOR ART

EP 0 704 207 A2 describes thermoplastic materials for pharmaceutical coatings which are soluble in intestinal juice. These are copolymers of 16 to 40% by weight acrylic or methacrylic acid, 30 to 80% by weight methyl acrylate and 0 to 40% by weight other alkyl esters of acrylic acid and/or methacrylic acid.

EP 0 704 208 A2 describes coating agents and binders for pharmaceutical coatings which are soluble in intestinal juice. These are copolymers of 10 to 25% by weight methacrylic acid, 40 to 70% by weight methyl acrylate and 20 to 40% by weight methyl methacrylate. The description mentions multilayer coating systems in addition to monolayer coatings. These systems may consist of a core which comprises for example a basic or a water-sensitive active ingredient, have a sealing layer of another coating material such as cellulose ether, cellulose ester or a cationic polymethacrylate, e.g. of EUDRAGIT® E, RS or RL type, and are then provided additionally with the abovementioned coating which is soluble in intestinal juice.

EP 0 519 870 A1 describes oral diclofenac preparations. The active ingredient is applied to a core provided with a bilayer coating. The inner layer may consist of a neutral (meth)acrylate copolymer of the EUDRAGIT® NE type and comprises, besides the pharmaceutically usual excipients such as, for example, mold release agents, from 5 to 20% by weight of a pore former, e.g. red iron oxide. The outer layer is resistant to gastric juice and may consist for example of a (meth)acrylate copolymer of the EUDRAGIT® L type.

U.S. Pat. No. 5,643,602 describes oral pharmaceutical forms for the therapy of ulcerative colitis or Crohn's disease. The pharmaceutical form has a multilayer structure with a neutral core inside and subsequently two polymer layers. The active ingredient in this case is present in an inner layer mixed with a neutral polymer, e.g. ethylcellulose or EUDRAGIT® NE. The outer layer is resistant to gastric juice and may consist for example of a (meth)acrylate copolymer of the EUDRAGIT® L type.

WO 01/68 058 describes a multilayer pharmaceutical form which is substantially composed of a) a core with an active pharmaceutical ingredients b) an inner coating of a copolymer or a mixture of copolymers which are composed of 85 to 98% by weight free-radical-polymerized C₁- to C₄-alkyl esters of acrylic or of methacrylic acid and 15 to 2% by weight (meth) acrylate monomers having a quaternary ammonium group in the alkyl radical, and c) an outer coating of a copolymer which is composed of 75 to 95% by weight free-radical-polymerized C₁- to C₄-alkyl esters of acrylic or of methacrylic acid and 5 to 25% by weight (meth) acrylate monomers having an anionic group in the alkyl radical.

WO 2004/039357 describes a multilayer pharmaceutical form composed of a) a neutral core, b) an inner coating of a methacrylate copolymer and c) an outer coating of a copolymer which is composed of 40 to 95% by weight free-radical-polymerized C₁- to C₄-alkyl esters of acrylic or of methacrylic acid and 5 to 60% by weight (meth)acrylate monomers having an anionic group in the alkyl radical. The pharmaceutical form is characterized in that the inner coating consists substantially of a methacrylate copolymer which is composed of at least 90% by weight of (meth)acrylate monomers having neutral radicals, has a minimum film-forming temperature as specified in DIN 53 787 not exceeding 30° C., and comprises the active pharmaceutical ingredient in bound form.

### PROBLEM AND SOLUTION

Pharmaceutical forms according to WO 01/68058 have excellent properties for the release of active ingredients in the colon. Virtually no active ingredient is delivered into the stomach, and a uniform and long-lasting delivery of active ingredient into the intestine, in particular shortly before or only in the colonic region, is achieved. The mode of delivery of the active ingredient is such as to comply with the in vitro requirement that in the USP release test two hours at pH 1.2 and subsequent change in the buffer to pH 7.0, the release of the active ingredient present is less than 5% in the period up to 2.0 hours after the start of the test and 30 to 80% at the time eight hours after the start of the test.

However, it has been found that the coatings of the described pharmaceutical form do not always have suitable mechanical properties. Especially in the case of very thin film coatings, e.g. with slightly soluble or high-dose medicinal substances, there is a need for increased mechanical strength to stabilize the film coatings in production processes customary in pharmaceuticals, such as compression, packing into capsules or sachets or mixing with other pellet preparations. Similar considerations apply to pharmaceutical forms disclosed in EP 0 519 870 A1 or WO 2004/039357.

The problem was therefore regarded as being to provide a pharmaceutical form with at least very similar release characteristics but which is improved in the mechanical properties of the film coating.

The problem is solved by a multilayer pharmaceutical form comprising
a) a core with an active pharmaceutical ingredient
b) an inner coating which consists of 50 to 95% by weight of a (co)polymer which is composed of 95 to 100% by weight of free-radical-polymerized vinylic monomers having neutral side groups and 0 to 5% by weight monomers having anionic side groups,
c) an outer coating of a copolymer which is composed of 75 to 95% by weight free-radical-polymerized C₁- to C₄-alkyl esters of acrylic or of methacrylic acid and 5 to 25% by weight (meth) acrylate monomers having an anionic group in the alkyl radical, where 5 to 30% by weight of pharmaceutically usual excipients, especially plasticizers, are present,
   characterized in that
the inner coating comprises 5 to 50% by weight of pharmaceutically usual excipients which are not pore formers, and pore formers are present only in amounts of less than 5% by weight.

The combination of the inner and outer coating film evidently lead to an increased tensile strength of the double film layer as a whole compared with WO 01/68058, The mechanical properties of the pharmaceutical form itself and of multiparticulate pharmaceutical forms produced therefrom are thus distinctly improved. The improvement in properties is identifiable on isolated double film layers. Tensile strengths in the range from 6 to 10 [Mpa] and nominal tensile strains at break in the range from 170 to 300 [%] are measured for isolated double film layers having the structure according to the invention.

### IMPLEMENTATION OF THE INVENTION

The invention relates to a multilayer pharmaceutical form comprising

Core a)

Carriers or cores for the coatings are tablets, granules, pellets, crystals of regular or irregular shape. The size of granules, pellets or crystals is ordinarily between 0.01 and 2.5 mm, and that of tablets between 2.5 and 30.0 mm. The carriers normally comprise 1 to 95% active ingredient and, where appropriate and usually, further pharmaceutical excipients.

The usual production processes are direct compression, compression of dry, moist or sintered granules, extrusion and subsequent rounding off, wet or dry granulation or direct pelleting (e.g. on plates) or by binding of powders (powder layering) on active ingredient-free beads (nonpareilles) or active ingredient-containing particles.

Besides the active ingredient, the cores may contain further pharmaceutical excipients: binders such as lactose, cellulose and derivatives thereof, polyvinylpyrrolidone (PVP), humectants, disintegration promoters, lubricants, disintegrants, starch and derivatives thereof, sugar solubilizers or others.

The cores a) can be provided in the usual way with an active pharmaceutical ingredient by applying the appropriate active ingredient for example as active ingredient powder to carrier particles (nonpareilles) by means of an aqueous hinder. The active ingredient cores (pellets) can be obtained after drying and screening in the desired size fraction (e.g. 0.7 to 1 mm). This process is referred to inter alia as powder layering. The active ingredient content of the core can be for example from 5 to 90% by weight.

Inner Coating b)

The inner coating b) consists of 50 to 95, preferably 60 to 90, % by weight of a (co) polymer which is composed of 95 to 100, preferably 98 to 100, % by weight of free-radical-polymerized vinylic monomers having neutral side groups and 0 to 5, preferably 0 to 2, % by weight vinylic monomers having anionic side groups. The copolymer is predominantly or completely neutral and preferably has the property of swelling in water above pH 5.0 or in the medium of intestinal juice, and releasing the active ingredient in controlled or sustained fashion.

The active ingredient release characteristics do not correspond exactly to those described in WO 01/68058, but the differences are surprisingly small. The modification in favor of better mechanical properties therefore appears to be perfectly tolerable. The release profile can be adapted where appropriate by varying the layer thickness of the inner coating.

The inner coating may comprise a (co)polymer which is composed of 95 to 100, preferably 98 to 100, % by weight free-radical-polymerized C₁- to C₄-alkyl esters of acrylic or of methacrylic acid and optionally 0 to 5, preferably 0 to 2, % by weight vinylic monomers having anionic side groups, in particular acrylic and/or methacrylic acid.

C₁- to C₄-Alkyl esters of acrylic or methacrylic acid are in particular methyl methacrylate, ethyl methacrylate, butyl methacrylate, methyl acrylate, ethyl acrylate and butyl acrylate.

A (meth)acrylate monomer having an anionic group in the alkyl radical may be for example acrylic acid, but preferably methacrylic acid.

Suitable examples are neutral (meth)acrylate copolymers composed of 20 to 40% by weight ethyl acrylate and 60 to 80% by weight methyl methacrylate (EUDRAGIT® NE type).

EUDRAGIT® NE is a copolymer of 30% by weight ethyl acrylate and 70% by weight methyl methacrylate.

The inner coating may comprise a (co)polymer which is polyvinyl acetate or a polyvinyl acetate, The expression “a polyvinyl acetate” includes derivatives of polyvinyl acetate. The polyvinyl acetate may be in the form of a dispersion (e.g. of the Kollicoat® SR. 30 D type, manufactured by BASF, polyvinyl acetate dispersion stabilized with povidone and Na lauryl sulfate).

The inner coating comprises 5 to 50% by weight of pharmaceutically usual excipients which are not pore formers.

It has been found that pore formers like those used in EP 0 519 870 A1 have adverse effects on the mechanical properties of the double coating film layer if they are present, as in EP 0 519 870 A1, in the inner layer. The inner coating layer may, even if this does not appear expedient, comprise a small amount of pore former without the mechanical properties of the double coating inevitably being too greatly impaired. Pore formers ought to be used in the inner coating only in amounts of less than 5, preferably less than 2 or 1, % by weight, or preferably not at all. Such small amounts normally have no technical effect. It is therefore particularly preferred for no pore formers to be present in the inner coating layer.

The pharmaceutically usual excipients which may be present in the inner coating are selected from the substance classes of plasticizers, stabilizers, colorants, antioxidants, wetting agents, pigments, gloss agents, mold release agents; antitack agents, with the content of pore formers, in particular water-insoluble pore formers such as kaolin, calcium carbonate, calcium hydrogen phosphate, magnesium oxide, microcrystalline cellulose, titanium dioxide or iron oxide, and especially water-soluble pore formers such as povidone K30, polyvinyl alcohol, cellulose derivatives such as hydroxypropylcellulose, hydroxypropylmethylcellulose (HPMC), methylcellulose or sodium carboxymethylcellulose, sucrose, xylitol, sorbitol, mannitol, maltose, xylose, glucose, potassium chloride, sodium chloride, polysorbate 80, polyethylene glycol or sodium citrate, being zero or only amounts of less than 5, preferably less than 2 or 1, % by weight.

It has further been found that an active ingredient bound in the inner coating layer, as suggested in WO 2004/039357, likewise has a disadvantageous effect on the mechanical properties of the double coating film layer. The active ingredient present in the pharmaceutical form is expediently accommodated in the core layer. The inner coating layer may, even if this does not appear expedient, comprise a small amount of active ingredient without the mechanical properties of the coating inevitably being too greatly impaired. The active ingredient content in the inner coating ought, however, to be less than 2, preferably less than 1. Such small amounts normally have no technical effect. It is therefore particularly preferred for no active ingredient to be present in the inner coating layer.

The layer thickness of the inner coating may be for example in the range 10-100, preferably from 20 to 40 μm.

Outer Coating c)

The outer coating c) comprises a copolymer which is composed of 75 to 95% by weight free-radical-polymerized C₁- to C₄-alkyl esters of acrylic or of methacrylic acid and 5 to 25% by weight (meth)acrylate monomers having an anionic group in the alkyl radical, with 5 to 30, preferably 8 to 20, % by weight of pharmaceutically usual excipients, in particular plasticizers, being present. Pore formers should be used in the outer coating only in amounts of less than 5, preferably less than 2 or 1, % by weight, or preferably not at all. Such small amounts normally have no technical effect. It is therefore particularly preferred for no pore formers' to be present, in the outer coating layer.

C₁₋C₄-Alkyl esters of acrylic or methacrylic acid are, in particular, methyl methacrylate, ethyl methacrylate, butyl methacrylate, methyl acrylate, ethyl acrylate and butyl acrylate.

A (math)acrylate monomer having an anionic group in the alkyl radical can be, for example, acrylic acid, but preferably methacrylic acid.

Particularly suitable (meth)acrylate copolymers are those composed of 10 to 30% by weight methyl methacrylate, 50 to 70% by weight methyl acrylate and 5 to 15% by weight methacrylic acid (EUDRAGIT® FS type).

The copolymers are commercially available and can be obtained in a manner known per se by free-radical bulk, solution, bead or emulsion polymerization. Before processing, they must be brought to the particle size range according to the invention by suitable grinding, drying or spraying processes. This can take place by simple crushing of extruded and cooled pellets or hot cut.

Preference is given to emulsion polymerization in aqueous phase in the presence of water-soluble initiators and (preferably anionic) emulsifiers (see, for example, DE-C 2 135 073).

The emulsion polymer is preferably produced and used in the form of a 10 to 50 percent by weight, in particular 30 to 40 percent, aqueous dispersion. Partial neutralization of the methacrylic acid units is not necessary for processing; it is, however, possible, for example to the extent of 5 or 10 mol %, if thickening of the coating agent dispersion is desired. The weight-average size of the latex particles is ordinarily 40 to 100 nm, preferably 50 to 70 nm, which ensures a viscosity of below 1000 mPa·s which is favorable for processing.

The layer thickness of the outer coating may be for example in the range 20-150, preferably from 40 to 80 μm.

Inner/outer Coating Amount Ratios

The total weight of the inner coating may preferably amount to 2 to 50, particularly preferably 10 to 40, % by weight based on the total weight of the core.

The total weight of the core is composed of the active ingredient, the excipients used where appropriate for the formulation, including neutral cores (nonpareilles) used where appropriate, and thus corresponds to the dry weight of the formulation.

The total weight of the inner coating is composed of the copolymer and the excipients present, and thus corresponds to the dry weight of the formulation used.

The total weight of the outer coating is composed of the copolymer and the excipients present where appropriate, e.g. plasticizer, and thus corresponds to the dry weight of the formulation used.

The total weight of the outer coating may preferably amount to 5 to 50, particularly preferably 10 to 30, % by weight based on the total weight of the core and of the inner coating.

Moreover, scanning electron micrographs of cross sections of isolated double films having the structure according to the invention show homogeneous, uniform layers with good adhesion at the interface.

Process

The invention further relates to a process for producing the pharmaceutical form of the invention, characterized by the steps
a) Production of a core having a pharmaceutical by means of spray application to a neutral core (nonpareilles) or by rotagglomeration, precipitation spray processes or extrusion and spheronization without a neutral core produces and subsequently,
b) application of the inner coating by spray application so that active ingredient-containing, coated pellets are obtained,
c) application of the outer coating by spray application so that active ingredient-containing, doubly coated pellets are obtained,
d) optionally a final curing treatment to stabilize the release profile, e.g. by storing in the dry at 40° C. for 2 hours.

The resulting pellets can be further processed with the aid of pharmaceutically usual excipients and in a manner known per se to give a multiparticulate pharmaceutical form, in particular pellet-containing tablets, minitablets, capsules, sachets or reconstitutable powders, which are formulated so that the contained pellets are released in the pH range of the stomach.

Multiparticulate Pharmaceutical Form

The pharmaceutical form of the invention, e.g. in pellet form, may advantageously be used as constituent of a multiparticulate pharmaceutical form. The improved mechanical properties prove to be particularly advantageous during processing in production processes customary in pharmaceuticals, such as compression, packing into capsules or sachets or mixing with other pellet preparations. The advantages emerge especially with very thin coatings and/or very high active ingredient loading. Particularly in the compression of pellets to tablets, where especially high mechanical forces occur, the pharmaceutical form of the invention proves to have low susceptibility to damage to the coating layers. The result is high process reliability and a great reproducibility of the properties of units from different production cycles.

Release Characteristics

Although the active ingredient release characteristics do not correspond exactly to those of WO 01/68058, they are similar. The differences are surprisingly small, The pharmaceutical form is therefore particularly suitable for release of active ingredients in the colon.

In the USP release test for two hours at pH 1.2 and a subsequent change in the buffer to pH 7.0, the release of the active ingredient present is less than 5% in the period up to 2.0 hours after the start of the test and 30 to 80%, in particular 40 to 70%, at the time eight hours after the start of the test.

The for example USP release test (according to USP XXIV, method B, modified test for enteric coated products) is known to the skilled worker. The test conditions are, in particular: paddle method, 100 revolutions per minute, 37° C.; pH 1.2 with 0.1 N HCl, pH 7.0 by addition of 0.2 M phosphate buffer and adjustment with 2 N NaOH. See also USP 27-NF22 Supplement 1, method “Delayed Release” monograph <724> Drug Release.

The multilayer pharmaceutical form to be used consists essentially of a core with an active ingredient, of an inner and of an outer coating. It is possible in the usual way for excipients in use in pharmacy to be present, but they are not critical for the invention.

Active Pharmaceutical Ingredients

The active pharmaceutical ingredients which can be employed for the purposes of the invention are intended to be used on or in the human or animal body in order
1. to heal, to alleviate, to prevent or to diagnose diseases, ailments, physical damage or pathological symptoms.
2. allow the state, the condition or the functions of the body or mental states to be identified.
3. to replace active substances produced by the human or animal body, or body fluids.
4. to defend against, to eliminate or to render innocuous pathogens, parasites or exogenous substances or
5. to influence the state, the condition or the functions of the body or mental states.

Drugs in use can be found in reference works such as, for example, the Rote Liste or the Merck Index. Examples which may be mentioned are 5-aminosalicylic acid, corticosteroids (budesonide), and proteins (insulin, hormones, antibodies). It is possible to employ according to the invention ail active ingredients which comply with the desired therapeutic effect within the meaning of the above definition and have an adequate stability and whose activity can be achieved via the colon in accordance with the above points.

Important examples (groups and single substances) without a claim to completeness are the following:

analgesics, antibiotics, antidiabetics, antibodies chemotherapeutics, corticoids/corticosteroids anti-inflammatory agents, enzyme products hormones and their inhibitors, parathyroid hormones peptic agents, vitamins, cytostatics

Active ingredients which should be particularly mentioned are those which are to be released as constantly as possible in the intestine, in particular shortly before or only in the colonic region. Thus, the active pharmaceutical ingredient may be an aminosalicylate, a sulfonamide or a glucocorticoid, in particular 5-aminosalicylic acid, olsalazine, sulfalazine, prednisone or budesonide.

### EXAMPLES OF ACTIVE INGREDIENTS

mesalazine

sulfasalazine

bethamethasone 21-dihydrogenophosphate

hydrocortisone 21-acetate

cromoglicic acid

dexamethasone

olsalazine Na

budesonide, prednisone

bismunitrate, karaya gum

methylprednisolone 21-hydrogen succinate

myhrr, coffee charcoal, camomile flower extract

10% suspension of human placenta

Newer Active Ingredients and Active Ingredients Under-going Development and Testing

(Literature from relevant pharmaceutical databases known to the skilled worker)

balsalazide

orally administered peptides (e.g. RDP 58)

interleukin 6

interleukin 12

ilodecakin (interleukin 10)

nicotine tartrate

5-ASA conjugates (CPR 2015)

monoclonal antibody against interleukin 12

diethyldihydroxyhomospermine (DEHOHO)

diethylhomospermine (DEHOP)

cholecystokinin (CCK) antagonist (CR 1795)

15 amino acid fragment of a 40 kd peptide from gastric

juice (BPC 15)

glucocorticoid analog (CBP 1011)

natalizumab

infliximab (REMICADE)

N-deacetylated lysoglycosphingolipid (WILD 20)

azelastine

tranilast

sudismase

phosphorothioate antisense oligonucleotide (ISIS 2302)

tazofelone

ropivacaine

5-lipoxygenase inhibitor (A 69412)

sucralfate

The pharmaceutical form may comprise an active pharmaceutical ingredient which is an enzyme, a peptide hormone, an immunomodulatory protein, an antigen or antibody.

The pharmaceutical form may comprise as active pharmaceutical ingredient a pancreatin, an insulin, a human growth hormone (hGH), corbaplatin, intron A, calcitonin, cromalyn, an interferon, a calcitonin, granulocyte colony stimulating factor (G-CSF), an interleukin, parathyroid hormones, glucagon, prosomatostatin, a somatostatin, detirelix, cetrorelix, vasopressin, 1-deaminocysteine-3-D-arginine-vasopressin, leuprolide acetate or an antigen which has been isolated from grasses or other plants such as, for example, rye, wheat, barley, oats, bermuda grass, horsetail, sycamore, elm, oak, plane tree, poplar, cedar, horsetail, thistles.

Pharmaceutically Usual Excipients

Pharmaceutically usual excipients for the purposes of the present invention exclude pore formers in proportions form 5% by weight, based on the inner coating.

To produce the multilayer pharmaceutical form it is possible to employ pharmaceutically usual excipients in the usual way.

Antitack agents (nonstick agents): Antitack agents have the following properties: they have large specific surface areas, are chemically inert, are free-flowing and comprise fine particles. Because of these properties, they reduce the tack of polymers containing polar comonomers as functional groups.

Examples of antitack agents are:

alumina, magnesium oxide, kaolin, talc, glycerol monostearate, magnesium stearate, silica (Aercsils), syloid, barium sulfate,

Mold Release Agents

Examples of mold release agents are:

esters of fatty acids or fatty amides, aliphatic, long-chain carboxylic acids, fatty alcohols and esters thereof, montan waxes or paraffin waxes and metal soaps; particular mention should be made of glycerol monostearate, stearyl alcohol, glycerol behenic acid ester, cetyl alcohol, palmitic acid, canauba wax, beeswax etc. The usual proportionate amounts are in the range from 0.05% by weight to 5, preferably 0.1 to 3, % by weight based on the copolymer.

Further pharmaceutically usual excipients Mention should be made here of, for example, stabilizers, colorants, antioxidants, wetting agents, pigments, gloss agents etc. They are used in particular as processing aids and are intended can be to ensure a reliable and reproducible production process and good long-term storage stability. Further pharmaceutically usual excipients may be present in amounts of from 0.001% by weight to 30% by weight, preferably 0.1 to 10% by weight, based on the copolymer.

Plasticizers; Substances suitable as plasticizers ordinarily have a molecular weight between 100 and 20 000 and contain one or more hydrophilic groups in the molecule, e.g. hydroxyl, ester or amino groups. Citrates, phthalates, sebacates, castor oil are suitable. Examples of suitable plasticizers are alkyl citrates, glycerol esters, alkyl phthalates, alkyl sebacates, sucrose esters, sorbitan esters, dibutyl sebacate and polyethylene glycols 4000 to 20 000 . Preferred plasticizers are tributyl citrate, triethyl citrate, acetyl triethyl citrate, dibutyl sebacate and diethyl sebacate. The amounts used are between 1 and 35, preferably 2 to 10, % by weight based on the respective polymer or copolymer.

Administration Forms

The described pharmaceutical form can be in the form of a coated tablet, in the form of a tablet composed of compressed pellets or in the form of pellets which are packed in a capsule, for example made of gelatin, starch or cellulose derivatives.

### EXAMPLES

Testing of mechanical properties of 1- and 2-layer film coatings produced by casting

EUDRAGIT® RS: Copolymer of 65% by weight methyl methacrylate, 30% by weight ethyl acrylate and 5% by weight 2-trimethylammoniummethyl methacrylate chloride.

EUDRAGIT® RL: Copolymer of 6% by weight methyl methacrylate, 30% by weight ethyl acrylate and 10% by weight 2-triemethylammoniummethyl methacrylate chloride.

EUDRAGIT® NE: Copolymer of 30% by weight ethyl acrylate and 70% by weight methyl methacrylate.

EUDRAGIT® FS: Copolymer of 65% by weight methyl acrylate, 25% by weight methyl methacrylate and 10% by weight methacrylic acid.
1st layer=corresponds to the inner coating film
in a pharmaceutical form of the invention
2nd layer=corresponds to the outer coating film
in a pharmaceutical form of the invention

Production of the Film-forming Formulations:

EUDRAGIT® FS 30 D formulation, 10% strength aqueous, produced from a 30% strength EUDRAGIT FS 30 D dispersion and 5% (based on the polymer) triethyl citrate (TEC), the dispersion is diluted to 10% with deionized water:

TEC and water were weighed into a 400 ml glass beaker and stirred on a magnetic stirrer at 400 rpm until the TEC dissolved to give a clear solution.

The amount of EUDRAGIT® FS 30 D which has been filtered through an approx. 0.1 to 0.2 mm metal screen is introduced into a 500 ml PE screw-top bottle and, while stirring with the magnetic stirrer at about 400±100 rpm, the aqueous TEC solution is added thereto. The formulation is stirred at this speed at room temperature in the closed bottle for at least 1-2 hours.

The 10% strength dispersion was stored in a refrigerator at 4-8° C. overnight and, the next day, stirred up shortly before casting on the plate.

EUDRAGIT® RS 30 D/RL 30D (1:1) formulation, 10% strength aqueous,

prepared from a mixture of in each case 30% strength EUDRAGIT® RS 30 D/RL 30D (1:1) dispersion and 20% (based on the polymer) triethyl citrate, the dispersion is diluted to 10% with deionized water:

TEC and water were weighed into a 400 ml glass beaker and stirred on a magnetic stirrer at 500 rpm until the TEC dissolved to give a clear solution.

The amount of EUDRAGIT® RS 30 D/RL 30D (1:1) dispersion which has been filtered through an approx. 0.1 to 0.2 mm metal screen is introduced into a mi PE screw- top bottle and, while stirring with the magnetic stirrer at about 400±100 rpm, the aqueous TEC solution is added thereto.

The formulation is stirred at this speed at room temperature in a closed bottle overnight.

EUDRAGIT® NE 30D formulation, 10% strength aqueous, prepared from a 30% strength EUDRAGIT(r) NE 30 D dispersion and diluted to 10% diluted with deionized water:

The amount of EUDRAGIT® NE 30 D which has been filtered through an approx. 0.1 to 0.2 mm metal screen is introduced into a 500 ml PE screw-top bottle and, while stirring with the magnetic stirrer at about 400±100 rpm, the water is added thereto.

The formulation is stirred at this speed at room temperature in a closed bottle overnight.

Polyvinyl acetate (Kollicoat® SR 30 D) formulation, 10% strength aqueous,

prepared from a 30% strength polyvinyl acetate dispersion, 10% (based on the polymer) propylene glycol and 3% (based on the polymer) Kollidon® 25, the dispersion is diluted to 10% with deionized water:

Propylene glycol and water were weighed into a 400 ml glass beaker and stirred on a magnetic stirrer at 500 rpm until the propylene glycol dissolved. Kollidon® 25 is then introduced while stirring at a speed of initially 300 and later 990 rpm, and stirring is continued until Kollidon 25 is wetted.

Lumps are then dissolved with the aid of an Ultraturrax stirrer by stirring at about 900 rpm for about 15 min. The clear solution is then left to stand at room temperature for 5 min for air bubbles to escape.

The amount of the polyvinyl acetate dispersion filtered through an approx. 0.1 to 0.2 mm metal screen is introduced into a 500 ml PE screw-top bottle and, while stirring with the magnetic stirrer at about 400±100 rpm, the aqueous propylene glycol-Kollidon® 25 solution is added thereto.

The formulation is stirred at this speed at room temperature in a closed bottle overnight.

Film Casting

Preparation of the casting plates:

Three layers of a 2 cm fabric adhesive tape are glued around the edge of glass plates 20 cm×20 cm in size to result in a surround about 1 mm in height and an inner casting area of about 256 cm².

The inner casting area of about 256 cm² of the glass plate is then painted once with a pressure-sensitive adhesive and partly dried with a hot-air blower.

An aluminum foil 20 cm×20 cm in size from TSCHELLIN is then glued on this tacky surface with the matt side upward, i.e. rolled out flat thereon or spread out flat as far as the corners using a kitchen scraper. (Thickness of aluminum foil=0.012 mm thick, sides=shiny/matt soft, matt side=lacquer laminated on colored biaxially stretched polypropylene film 0.03 mm).

The aluminum foil which does not stick over the edge is curved upwards to result in an elevated surround area which is able to prevent the liquid running over.

The glass casting plates prepared in this way are then placed horizontally balanced with a spirit level in a convection drying oven.

Production, of 2- layer films:

All the produced formulations are filtered through an approx. 0.1 to 0.2 mm metal screen in each case before casting to produce the films.

64 g of a 10% strength EUDRAGIT® FS 30 D formulation which has been filtered through a metal screen are cast per plate as 1st ground layer on the glass casting plates which have been prepared and balanced in the convection drying oven at room temperature. Only then is the convection drying oven heated to 50° C., and the films are dried at this temperature with the fan at the minimum speed and an air flap 30% open for at least 3 days.

The FS 30 D films which now appear clear and are partially flat are then cooled to room temperature in the opened convection drying oven before the 2nd film layer is cast.

For each EUDRAGIT® and competing product sample, 3 glass casting plates are used with EUDRAGIT(r) FS 30 D films as 1st ground layer. 64 g of a 10% strength EUDRAGIT® or other sample filtered through a metal screen are then cast in each case on this EUDRAGIT(r) FS 30 D film ground layer.

In these cases too, the convection. drying oven is heated to 50° C. only after casting of the formulations, and the films are dried at this temperature with the fan at minimum speed and an air flap 30% open for at least 3 to 5 days until the films acquire a clear appearance, exception: 2-layer film with polyvinyl acetate (Kollicoat® SR 30 D) shows a slightly yellowish milky cloudiness (drying for 5 days) and with ethylcellulose (Aquacoat® ECD-30) shows a slightly cracked cloudiness with problems of adhesion to the underneath film (drying for 3 days).

The 2-layer films now obtained are cooled to room temperature, cautiously detached from the aluminum foil and stored separately in filter paper shaped pouches which are in turn sealed in a PE bag.

Production of 1-layer films:

All the produced formulations are filtered through an approx. 0.1 to 0.2 mm metal screen in each case before

casting to produce the films.

100 g of a 10% strength EUDRAGIT(r) or competing product formulation filtered through a metal screen, or 67 g of a 15% strength formulation (e.g. colloidal solution of formulation) filtered through a metal screen are cast in each case on 2 plates for each sample on the glass casting plates which have been prepared and balanced in the convection drying oven at room temperature. Only then is the convection drying oven heated to 50° C., and the films are dried at this temperature with the fan at minimum speed and an air flap 30% open for at least 3 days. After this time, the films of the EUDRAGIT® FS 30 D and EUDRAGIT® NE 30 D formulation show a clear appearance, Aquacoat® ECD-30 results in a very brittle film which shatters even on handling and thus cannot be determined. Film formulations of Kollicoat® SR 30 D and EUDRAGIT® RS 30 D/RL 30 D (1:1) can be heat treated again at 60° C. overnight after 3 days to remove the residual moisture. Attention must be paid to blistering in this case. The appearance with EUDRAGIT® RS 30 D/RL 30 D (1:1) is then clear or with minimal cloudiness, and with Kollicoat® SR 30 D is yellowish and cloudy.

The 1-layer films now obtained are cooled to room temperature, carefully detached from the aluminum film and stored separately in filter paper shaped pouches which are in turn sealed in a PE bag.

Tensile Testing:

Method: ISO 527-2/1BA/20

Test conditions: 23° C./50% R.H.

Chuck: Air

Machine: 1% accuracy class

Displacement sensor: Traverse

Length clamped: 57.5 mm

Conditioning: Standard conditions

(23° C./50% R.H.) for 16 h

Length: 57.5 mm

Preload: 0.05 MPa

### Example 1-10

| | | | | Nominal |
|---|---|---|---|---|
| | | | Tensile | tensile |
| | | | strength | strain at |
| Ex. | | Polymers | [Mpa] | break [%] |
| 1 | not according to | 1 layer of | 10.1 | 187 |
| | the invention | EUDRAGIT ® FS | | |
| 2 | not according to | 1 layer of | 1.5 | 257 |
| | the invention | EUDRAGIT ® | | |
| | | RL/RS (1:1) | | |
| 4 | not according to | 1 layer of | 4.1 | 819 |
| | the invention | EUDRAGIT ® NE | | |
| 5 | not according to | 1 layer of | 10.0 | 450 |
| | the invention | polyvinyl | | |
| | | acetate | | |
| 6 | not according to | 1st layer: | 5.4 | 174 |
| | the invention | EUDRAGIT ® | | |
| | (according to | RL/RS (1:1) | | |
| | WO 01/68058) | 2nd layer of | | |
| | | EUDRAGIT ® FS | | |
| 7 | not according to | 1st layer: | Cannot be determined | |
| | the invention | ethylcellulose | because the layers | |
| | | 2nd layer of | separate even during | |
| | | EUDRAGIT ® FS | preparation of the samples | |
| 9 | according to | 1st layer of | 7.0 | 174 |
| | the invention | EUDRAGIT ® NE | | |
| | | 2nd layer of | | |
| | | EUDRAGIT ® FS | | |
| 10 | according to | 1st layer of | 8.0 | 288 |
| | the invention | polyvinyl | | |
| | | acetate | | |
| | | 2nd layer of | | |
| | | EUDRAGIT ® FS | | |

It is evident from the measurements that ail the two-layer polymer systems reduce the strength, which is good per se, of a EUDRAGIT® FS layer. This effect is particularly strong with the combination according to Example 6 of WO 01/68058.

Scanning electron micrographs of cross sections of the films show homogeneous, uniform layers with good adhesion at the interface for all the double-layer films of the invention.

1. A multilayer pharmaceutical form comprising
a) a core with an active pharmaceutical ingredient,
b) an inner coating which consists of 50 to 95% by weight of a (co)polymer which is composed of 95 to 100% by weight of free-radical-polymerized vinylic monomers having neutral side groups and 0 to 5% by weight of monomers having anionic side groups, and
c) an outer coating of a copolymer which is composed of 75 to 95% by weight free-radical-polymerized C₁- to C₄-alkyl esters of acrylic or methacrylic acid and 5 to 25% by weight (meth)acrylate monomers having an anionic group in the alkyl radical, wherein 5 to 30% by weight of pharmaceutically usual excipients are present,
characterized in that
the inner coating comprises 5 to 50% by weight of pharmaceutically usual excipients which are not pore formers, and pore formers are present only in amounts of less than 5% by weight.

2. The pharmaceutical form as claimed in claim 1, characterized in that the inner coating comprises a (co)polymer which is composed of 95 to
100% by weight of free-radical-polymerized C₁- to C₄alkyl esters of acrylic or methacrylic acid and, where appropriate. 0 to 5% by weight acrylic or methacrylic acid.

3. The pharmaceutical form as claimed in claim 1, characterized in that the inner coating comprises a (co)polymer which is a polyvinyl acetate.

4. The pharmaceutical form as claimed in claim 1, characterized in that the pharmaceutically usual excipients in the inner coating are selected from the substance classes consisting of plasticizers, stabilizers, colorants, antioxidants, wetting agents, pigments, gloss agents, mold release agents, and antitack agents, with the content of pore formers selected from the group consisting of kaolin, calcium carbonate, calcium hydrogen phosphate, magnesium oxide, microcrystalline cellulose, titanium dioxide, iron oxide, povidone K30, polyvinyl alcohol, hydroxypropylcellulose, hydroxypropylmethylcellulose (HPMC), methylcellulose sodium carboxymethylcellulose, sucrose, xylitol, sorbitol, mannitol, maltose, xylose, glucose, potassium chloride, sodium chloride, polysorbate 80, polyethylene glycol and sodium citrate, being zero or only amounts of less than 5% by weight.

5. The pharmaceutical form as claimed in claim 1, characterized in that the total weight of the inner coating amounts to 2 to 50% by weight based on the total weight of the core.

6. The pharmaceutical form as claimed in claim 1, characterized in that the total weight of the outer coating amounts to 5 to 50% by weight based on the total weight of the core and the inner coating.

7. The pharmaceutical form as claimed in claim 1, characterized in that the contained active pharmaceutical ingredient is selected from the group consisting of an aminosalicylate, a sulfonamide and a glucocorticoid.

8. The pharmaceutical form as claimed in claim 7. characterized in that the active pharmaceutical ingredient is selected from the group consisting of 5-aminosalicylic acid, olsalazine, sulfalazine, prednisone and budesonide.

9. The pharmaceutical form as claimed in claim 1, characterized in that the active pharmaceutical ingredient is selected from the group consisting of an enzyme, a peptide hormone, an immunomodulatory protein, an antigen and an antibody.

10. The pharmaceutical form as claimed in claim 5, characterized in that the active pharmaceutical ingredient is selected from the group consisting of a pancreatin, an insulin, a human growth hormone (hGH), corbaplatin, intron A, calcitonin, cromalyn, an interferon, a calcitonin, granulocyte colony stimulating factor (G-CSF), an interleukin, parathyroid hormones, glucagon, pro-somatostatin, a somatostatin, detirelix, cetrorelix, vasopressin. 1-deaminocysteine-8-D-arginine-vasopressin, leuprolide acetate and an antigen which has been isolated from grasses or other plants.

11. A process for producing a pharmaceutical form as claimed in claim 1, characterized by the steps
a) production of a core having a pharmaceutical by means of spray application to a neutral core (nonpareilles) or by rotagglomeration, precipitation, spray processes or extrusion and spheronization without a neutral core and subsequently,
b) application of the inner coating by spray application so that active ingredient-containing, coated pellets are obtained,
c) application of the outer coating by spray application so that active ingredient-containing, doubly coated pellets are obtained, and
d) optionally a final curing treatment to stabilize the release profile of said doubly coated pellets by storing in the dry at 40° C. for 2 hours.

12. The process as claimed in claim 11, characterized in that the resulting pellets are processed with the aid of pharmaceutically usual excipients and in a manner known per se to a multiparticulate pharmaceutical form selected from the group consisting of pellet-containing tablets, minitablets, capsules, sachets and reconstitutable powders which are formulated so that the contained pellets are released in the pH range of the stomach.

13. A method of using a pharmaceutical form as claimed in claim 1 as a constituent of a multiparticulate pharmaceutical form.

14. The method as claimed in claim 13 wherein the form is used as a constituent of compressed tablets, capsules, sachets and reconstitutable powders.
